(19) United States
(12) Patent Application Publication
(10) Pub. No.: US 2019/0209047 A1
(43) Pub. Date: Jul. 11, 2019

(54) DEVICES, SYSTEMS AND METHODS FOR ENHANCED VISUALIZATION OF THE ANATOMY OF A PATIENT

(71) Applicant: Rox Medical, Inc., San Clemente, CA (US)

(72) Inventors: Brad KELLERMAN, San Clemente, CA (US); J. Christopher FLAHERTY, Topsfield, MA (US)

(21) Appl. No.: 16/354,911

(22) Filed: Mar. 15, 2019

Related U.S. Application Data
(63) Continuation of application No. 12/905,412, filed on Oct. 15, 2010.
(60) Provisional application No. 61/256,140, filed on Oct. 29, 2009.

(51) Int. Cl. A61B 5/107; A61B 8/08

(52) U.S. Cl. CPC A61B 5/1075; A61B 6/12; A61B 8/0841

(57) ABSTRACT

Devices, systems and methods are described for visualizing the anatomy of a patient. An expanding portion is configured to expand towards the tissue walls of a body space and be visible with one or more visualization instruments. Systems and methods are described which advance a probe from a first vessel toward a target in a second vessel.

### CROSS-REFERENCES TO RELATED APPLICATIONS

The present application is a continuation of U.S. patent application Ser. No. 12/905,412, filed Oct. 15, 2010, which claims the benefit of U. S. Provisional Application No. 61/256,140, filed Oct. 29, 2009, the full disclosures of which are incorporated herein by reference.

### BACKGROUND OF THE INVENTION

### Field of the Invention

The present invention relates to devices, systems and methods for enhancing the visualization of a location within a patient's body that is surrounded by one or more tissue walls, hereinafter a “body space”. Devices with expanding portions locate tissue walls and are visualized with standard imaging equipment. More particularly, the present invention relates to a system for advancing a probe from a first body space, such as a first vessel, toward a second body space, such as a second vessel. Patients include human beings as well as other mammalian species.

Numerous medical procedures require the visualization and/or measurement of a body space such as the space inside the stomach, a chamber of the heart or the lumen of a blood vessel. Imaging systems such as those using X-ray or ultrasound may be insufficient by themselves in providing accurate size and relative position information to a clinician performing a medical procedure.

Procedures including the implantation of a medical device often require three-dimensional body space information in order to properly select or size the implant. Procedures involving the access of a body space, such as the accessing of a lumen of a blood vessel from another blood vessel, require information regarding the specific location and orientation of the target vessel walls and lumen.

For these and other reasons, there is a need for devices, systems and methods which provide enhanced visualization of body spaces of a patient. Desirably the devices will be minimally invasive and have little or no side effects for the patient.

### BRIEF SUMMARY OF THE INVENTION

According to a first aspect of the invention, an anatomy visualization device is disclosed. The device includes an elongate filament with an expandable portion. The expandable portion is configured for insertion into a body space of a patient, such as an artery or vein of a patient. The body space is surrounded by one or more tissue walls. The expandable portion is further configured to radially expand to the one or more tissue walls. The expandable portion includes one or more markers, and/or is constructed of material that can be visualized by a visualization instrument such as a fluoroscope or other x-ray visualization apparatus; an ultrasound visualization apparatus; a CT-scanner; a magnetic resonance imaging apparatus (MM); a positron emission tomography (PET) scanner; an electromagnetic (EM) field detection apparatus; and combinations of these. The expanded portion material and/or markers may include one or more of: radiopaque material; electromagnetic components; magnets; ultrasonically reflective material; and/or other material configured to be visualized with one or more visualization instruments configured to visualize material within a body of a patient. A predetermined visualizable portion or marker size may be used to allow a clinician a reference to measure one or more structures in images taken. The one or more structures to be measured may be device structures and/or anatomical structures. Two or more markers may be placed on the visualization device with a known distance of separation.

The anatomy visualization device can be used to provide real time anatomical information, such as the location, shape, size and other geometric information related to a body space or the tissue walls of a body space. These types of information can be useful in numerous clinical procedures performed on a patient, such as information including but not limited to: vessel geometry information such as diameter, curvilinear shape and other vessel geometry information useful in an angioplasty, stenting, atherectomy and other vessel diagnostic or therapeutic procedures; fistula and intended fistula site information such as information regarding a preferred location for a fistula to be created and/or a needle or other probe to be advanced from a first vessel to a second vessel such as to a create a fistula during a TIPS procedure or a cardiopulmonary therapy procedure. A visualization instrument may be included to visualize the expanded portion. The visualization instrument may be selected from the group consisting of: a fluoroscope or other x-ray visualization apparatus; an ultrasound visualization apparatus; a Ct-scanner; a magnetic resonance imaging apparatus (MRI); a PET scanner; an electromagnetic (EM) field detection apparatus; and combinations of these.

The elongate filament may have a flexible construction such as a guidewire construction configured to navigate the vasculature of a patient. The elongate filament may be constructed of one or more biocompatible materials, such as Nitinol, stainless steel, and/or one or more polymers, and may include a coating or covering such as a hydrophobic, hydrophilic or polytetrafluoroethylene (PTFE) coating and/or a PTFE covering. The elongate filament may include a lumen from its proximal end to its distal end, such as to allow a guidewire, mandrel or other device to be inserted therethrough. A spiral or otherwise curved mandrel can be used to cause the expandable portion of the elongate filament to expand toward one or more of the patient's body space tissue walls. A sheath, including a distal end, may surround the expandable portion, such that longitudinal advancement of the expanded portion or retraction of the sheath causes the expandable portion to exit the distal end of the sheath. The expandable portion may be resiliently biased such that as exiting the distal end of the sheath, the expandable portion transitions from a constrained condition to an expanded condition. The expandable portion may include a single filament, such as a filament which is in a helical spiral when expanded. The expandable portion may include two or more filaments, such as multiple tines which are configured to radially expand, such as when a surrounding sheath is manipulated to expose the multiple tines.

The expandable portion expands to one or more tissue walls of a body space of a patient. A clinician may visualize the expanded portion and use the image as a target for advancing a probe, or for performing one or more other medical events or diagnostic assessments. The expandable portion may include one or more shape memory materials. The shape memory materials may be configured to expand due to a temperature change, such as a change from room temperature to body temperature. The shape memory materials may expand when heated, such as by passing a current through a resistive shape memory material. The expandable portion may be configured to be mechanically activated, such as via contraction by a pull wire, or insertion of a shaped mandrel such as a mandrel elastically biased in a helical spiral shape that is inserted into a linear elongate filament. The expandable portion may be magnetically or electromagnetically expanded.

The anatomy visualization device may be configured to provide structural support to one or more tissue walls, such as the expandable portion providing radial support configured to prevent collapse of the tissue walls. The expandable portion may be configured to be constrained, compacted or otherwise unexpanded, such as to allow removal from the patient's anatomy. The anatomy visualization device may be configured to enter arteries and/or veins of the patient, as well as other body spaces including but not limited to: a chamber of the heart; the stomach; the urethra; the binary duct; and other body cavities.

The anatomy visualization device may include a handle on its proximal end, such as a handle with one or more controls. A control may be configured to perform one or more operations, such as an operation selected from the group consisting of: advance or retract a filament; cause radial expansion or contraction; deliver energy such as energy delivered to a fistula site; apply positive pressure or vacuum; and combinations of these. The handle may include one or more markings. The markings may be visual and/or tactile markings. The markings may provide information to the operator such as information related to: advancement or retraction of a filament; amount of expansion of a visualization device expandable portion such as the amount of radial expansion; amount of force applied to tissue walls by a visualization device; amount of advancement or retraction of a probe such as a needle; and combinations of these.

According to another aspect of the invention, a system for advancing a probe from a first vessel into a second vessel at a target location in a patient is disclosed. The system includes a probe advancement device and an anatomy visualization device. The probe advancement device includes an elongate tube with a proximal end and a distal end, and an advanceable probe. The probe advancement device is configured to be placed intraluminally in a first vessel. The anatomy visualization device includes a target portion and is configured to be placed intraluminally in a second vessel. The probe of the probe advancement device is configured to be advanced from the first vessel toward the target portion of the anatomy visualization device, and into the second vessel. The probe may exit the distal end of the elongate tube or through a side hole proximal to the distal end. The probe may comprise a needle or other hollow tube configured to penetrate through tissue toward a target. The probe may deliver a separate device, such as a guidewire, or may deliver a therapeutic agent such as a pharmaceutical agent.

The first vessel and second vessel may be arteries or veins. In a preferred embodiment, one of the vessels is an artery selected from the group consisting of: femoral artery; internal iliac artery; external iliac artery; subclavian artery; and the aorta. In another preferred embodiment, one of the vessels is a vein selected from the group consisting of: femoral vein; internal iliac vein; external iliac vein; subclavian vein; and the inferior vena cava. The target location may be an intended location for a fistula to be created, such as a fistula created over a guidewire placed through the probe of the probe advancement device. The fistula may be a therapeutic fistula, such as a fistula created to treat one or more of: chronic obstructive pulmonary disease (COPD); congestive heart failure; heart failure; hypertension; hypotension; coronary artery disease; respiratory failure; lung fibrosis; adult respiratory distress syndrome (ARDS); chronic bronchitis; emphysema; cystic fibrosis; cystic lung disease; and chronic asthma. Alternatively or additionally, the fistula may be created to allow continued removal or administration of blood, such as is needed in a dialysis procedure. Alternatively or additionally, the fistula may be used to deliver a drug or other agent from one vessel to another vessel.

The system may include a dilation device, such as a balloon integral to the probe advancement device. The system may include an anastomotic clip, such as an anastomotic clip delivered by a delivery catheter or the probe advancement device. The system may include a device configured to snare or otherwise capture the probe advancement device probe, once the probe has been advanced into the second vessel. The probe capture device may be integral to the anatomy visualization device, such as when the anatomy visualization device includes a collapsible cage configured to capture the advanced probe. The capture device can be configured to retract the advanced probe, or a guidewire or other filament advanced through the probe, proximal in the second vessel, distal in the second vessel, or both proximal and distal.

According to yet another aspect of the invention, a method of advancing a probe from a first vessel to a second vessel at a target location in a patient is disclosed. A probe advancement device is placed into the first vessel. The probe advancement device includes an elongate tube with a proximal end and a distal end, and an advanceable probe. An anatomy visualization device, including a target portion, is placed into the second vessel. The target portion of the anatomy visualization device is advanced intraluminally to a target location of the patient's anatomy. The probe of the probe advancement device is transluminally advanced toward the target portion and into the second vessel. The probe advancement device may be intraluminally advanced in the first vessel, prior to advancing the probe into the second vessel. After the probe is advanced into the second vessel, the visualization device can be removed, while maintaining access of the probe into the second vessel. Alternatively or additionally, a guidewire or other filament can be placed through the probe into the second vessel, and the visualization device removed while maintaining access to the second vessel by the guidewire or other filament. The anatomy visualization device may be configured to capture the advanced probe, or a device advanced through the probe. The capturing portion may include a collapsible basket configured to snare the probe or other filament passing from the first vessel into the second vessel.

### BRIEF DESCRIPTION OF THE DRAWINGS

The accompanying drawings, which are incorporated in and constitute a part of this specification, illustrate various embodiments of the present invention, and together with the description, serve to explain the principles of the invention. In the drawings:

FIG. 1a illustrates a side sectional view of an anatomy visualization device in an unexpanded state and inserted into a body space, consistent with the present invention;

FIG. 1b illustrates the anatomy visualization device of Fig. la in an expanded state;

FIG. 2a illustrates a side sectional view of a probe advancement system including an anatomy visualization device including a spiral portion and inserted into an artery, and a probe advancement device inserted into a neighboring vein, consistent with the present invention;

FIG. 2b illustrates the system of FIG. 2a with a probe advanced into the artery;

FIG. 2c illustrates the system of FIGS. 2a and 2b, with a guidewire advanced through the probe and down the lumen of the artery;

FIG. 3 illustrates a side sectional view of a probe advancement system including an anatomy visualization device including an expandable cage and inserted into an artery, and a probe advancement device inserted into a neighboring vein, consistent with the present invention;

FIG. 4a illustrates a side sectional view of a probe advancement system including an anatomy visualization device including a lumen and inserted into an artery, and a probe advancement device inserted into a neighboring vein, consistent with the present invention;

FIG. 4b illustrates the system of FIG. 4a further including an imaging apparatus and a spiral mandrel that has been inserted into the lumen of the anatomy visualization device;

FIG. 5 illustrates a side sectional view of a probe advancement system including the anatomy visualization device of FIG. 2a inserted into an artery; and a probe advancement device including a catheter and advanced from the neighboring vein into the artery, consistent with the present invention;

FIG. 6 illustrates a side sectional view of a probe advancement system including the anatomy visualization device of FIG. 2a inserted into an artery; and a probe advancement device including a radiation delivery device and advanced from the neighboring vein into the artery, consistent with the present invention.

FIG. 7 illustrates a perspective view of an anatomy visualization device with an open cell design, consistent with the present invention.

### DETAILED DESCRIPTION OF THE INVENTION

Reference will now be made in detail to the present embodiments of the invention, examples of which are illustrated in the accompanying drawings. Wherever possible, the same reference numbers will be used throughout the drawings to refer to the same or like parts.

Visualization Instruments such as X-Ray units, fluoroscopes, ultrasound imagers, CT-scanners, PET scanners, and magnetic resonance imagers (MRIs) provide historic and real time imaging of a patient's anatomy. These images are used by a clinician performing one or more medical procedures on the patient. In addition to use in patient diagnosis, these images are often used to size a medical device such as a tool or an implant, or to navigate the patient's anatomy, such as in an interventional or surgical procedure. The anatomy visualization devices of the present invention provide additional information to those images. The devices of the present invention may be used to measure the diameter of a vessel, such as for an angioplasty or stenting procedure. The devices of the present invention can be used to enhance navigation through the body, such as with real-time visualization information used to manipulate a needle or other probe to penetrate a vessel, preferably toward the visualization device. Such procedures include percutaneous fistula creation procedures and transjugular intrahepatic portosystemic shunt (TIPS) procedures. The devices of the present invention can be used in percutaneous procedures, such as procedures which enter the body through an access sheath, or a surgical procedure such as a minimally invasive or laparoscopic surgical procedure.

The systems and methods of the present invention are used to visualize a body space, and also to access that body space from another location such as a second body space. In a preferred embodiment, the first body space is a blood vessel such as an artery, and the second body space is second blood vessel such as a vein. Such access can be used to place a guidewire, over which one or more tools can be placed. These tools may include a needle or catheter such as to deliver agents such as drugs. These tools may include a therapeutic probe, such as a flexible probe with a radiation delivery element at its tip. These tools may also include dilation devices and/or anastomosis devices such as to create a fistula between the two body spaces. The fistula may be created to provide a dialysis access site, or the fistula may provide the therapy. Methods and devices for performing arteriovenous fistula therapy (AVF), are described in the following co-pending applications, each of which is incorporated in its entirety herein by reference: Ser. Nos. 10/820,169; 11/961,731; 11/152,284; 11/013,981; 11/152,621; 11/151,802; 11/282,341; 11/356,876; 11/696,635; 11/946,454; and 12/017,437.

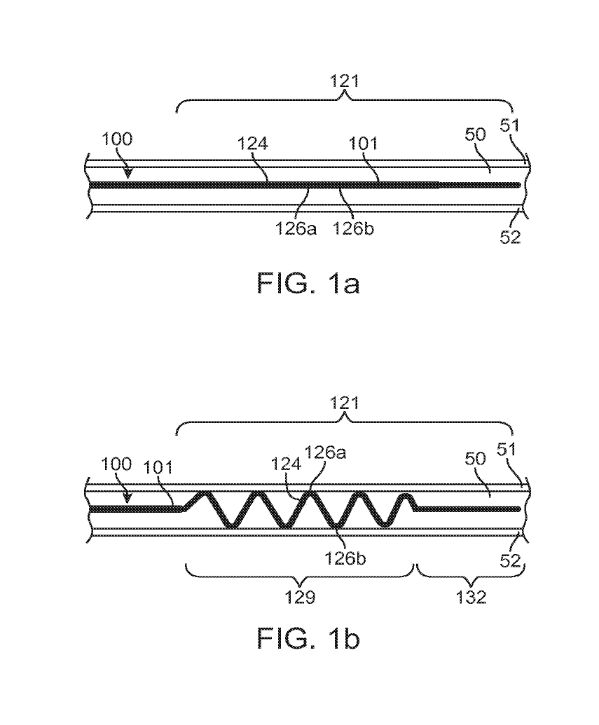

Referring to FIG. 1a, an anatomy visualization device of the present invention is illustrated. Visualization device 100 is inserted into body space 50, such as the stomach cavity, a chamber of the heart, a lumen of a blood vessel, the urethra, the esophagus, or the biliary duct. Body space 50 includes first wall 51 and second wall 52. One or more portions of visualization device 100 are visible in the images provided by a visualization instrument. Visualization instruments commonly found in hospitals and other health care centers include but are not limited to: X-rays and fluoroscopes; ultrasound imagers; CT-scanners; PET scanners; and other non-invasive or minimally invasive visualization instruments. Visualization device 100 includes sheath 101 which is in an advanced position such that distal end of helical filament 124 is constrained within sheath 101. Filament 124 is flexible, preferably of guidewire construction well known to those of skill in the art, and includes distal portion 121. Filament 124 is made of a biocompatible material such as Nitinol, stainless steel, cobalt chrome and/or a polymer or a polymer blend. Filament 124 may include one or more coatings or coverings, such as a hydrophilic or hydrophobic coating, or a polytetrafluoroethylene (PTFE) coating or covering. Filament 124 includes two markers, 126a and 126b, preferably radiopaque markers but alternatively markers such as ultrasonically reflective and/or magnetic markers. In a preferred embodiment, distal portion 121 is radiopaque and markers 126a and 126b provide a darker image than distal portion 121 in an X-ray. Alternatively or additionally, one or more portions or the entirety of device 100 may be covered by a radiopaque coil or material, such as coverings of different radiopacity which are used to distinguish different portions of device 100.

Referring now to FIG. 1b, the visualization device 100 of FIG. 1a is shown with sheath 101 retracted such as via a control on a handle, control and handle not shown but preferably on the proximal end of visualization device 100. After sheath 101 is refracted, distal portion 121 radially expands. In a preferred embodiment, distal portion 121 is elastically biased in the helical spiral shown, such that retraction of sheath 101 causes distal portion 121 to transition from its near linear constrained condition shown in FIG. 1a, to the expanded helical geometry shown in FIG. 1b. In an alternative embodiment, distal portion 121 may be temperature activated, such as via a body temperature activated shaped memory alloy. In another alternative embodiment, distal portion 121 is activated by a mechanical mechanism such as an internal pullwire or inserted shaped mandrel. Distal portion 121 has expanded sufficiently to engage wall 51 and wall 52 of body space 50, such that an image of distal portion 121 will indicate the position of walls 51 and 52, which may be invisible or difficult to visualize with the imaging technology used. As shown in FIG. 1b, markers 126a and 126b are positioned such as to contact opposing walls, such as opposing points along the wall of a blood vessel. The length of marker 126a and/or 126b can be used to measure a part of the patient's anatomy, such as via a comparative measurement. Alternatively or additionally, the distance between marker 126a and 126b may be used as a measuring tool. Distal portion 121 is shown with a multiple turn helix. In alternative embodiments, a partial helix (i.e. less than 360 degrees) may be employed. Distal portion 121 is configured to radially collapse, such as through advancement of sheath 101, and be atraumatically withdrawn from the patient. The diameter of the helix of distal portion 121 is sized based on the anatomy in which visualization device 100 is intended to visualize. The spiral section 121 is typically oversized 1-2 mm over the body space where the device is intended to be deployed. For a device intended to be placed in the iliac artery its expanded size is preferably between 8 and 10 mm. However, the diameter could be any size, typically from 2 to 25 mm. The length of the helix of distal portion 121 is typically 30 mm, preferably between 10 and 60 mm. Distal to target portion 129 is straight portion 132. In one embodiment, straight portion 132 is of similar diameter and construction to target portion 129. In an alternative embodiment, target portion has a smaller diameter than straight portion 132, such as to reduce the radial force exerted by target portion 129 upon walls 51 and 52, while maintaining the trackability of visualization device 100. Straight portion 132 typically has a length of 1 to 10 cm, preferably 4 to 7 cm and may be constructed similar to an interventional guidewire.

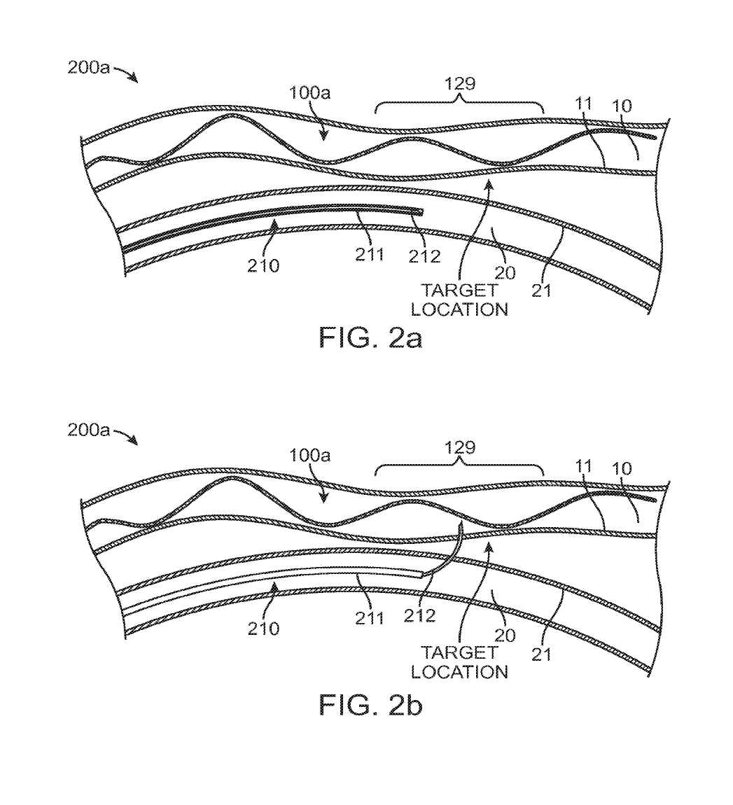

Referring now to FIGS. 2a through 2c, a probe advancement system of the present invention is illustrated. Probe advancement system 200a includes visualization device 100a and probe advancement device 210. Visualization device 100a has been placed in artery 10, and probe advancement device 210 has been placed in vein 20, each such as via a percutaneous vessel access sheath common to interventional medical procedures. A Target Location is chosen for advancement of a probe from vein 20 to artery 10. One reason for choosing the Target Location shown is the proximity of artery 10 to vein 11 at the target location. As is illustrated, artery 10 and vein 11 diverge from each other away from the Target Location, such that the Target Location will have the shortest length fistula within the region shown. The visualization devices and probe advancement devices of the present invention, including visualization device 100a and probe advancement device 210, include one or more visualizable portions (e.g. radiopaque portions, ultrasonically reflective portions, electromagnetically visible portions, etc.), such that positioning of the appropriate portions of visualization device 100a and probe advancement device 210, can be performed with standard visualization instruments such as fluoroscopy. In a preferred embodiment, an advanceable probe is radiopaque and a distal target portion of the visualization device is radiopaque, such that the probe can be advanced toward the target portion.

In an alternative embodiment, a probe is advanced from an artery to a vein. In a preferred embodiment, a fistula is to be created at the Target Location. Alternatively or additionally, the Target Location may be chosen to deliver drugs into artery 20 or deliver radiation in artery 20 at the Target Location. A fistula may be created to provide an access site for dialysis. A fistula may alternatively be created for therapeutic shunting of arterial blood into the venous system, such as to treat a cardiopulmonary disease or disorder. Cardiopulmonary conditions applicable to the devices, systems and methods of the present invention include but are not limited to: chronic obstructive pulmonary disease (COPD); congestive heart failure; heart failure; hypertension; hypotension; coronary artery disease; respiratory failure; lung fibrosis; adult respiratory distress syndrome (ARDS); chronic bronchitis; emphysema; cystic fibrosis; cystic lung disease; and chronic asthma ; and combinations of these. Visualization device 100a preferably includes a handle on its proximal end, not shown but typically including one or more controls such as to deploy the spiral at its distal end. Probe advancement device 210 also preferably includes a handle on its proximal end, not shown but typically including one or more controls such as to advance a probe at its distal end. Handles of the present invention may include multiple controls to activate or control multiple functions from outside the skin of the patient, such as to advance or retract a filament, cause radial expansion or contraction, deliver energy such as energy delivered to a fistula site; apply positive pressure or vacuum, or other functions common to interventional medical devices. Handles of the present invention may include markings or other visual, tactile or other feedback to allow an operator to precisely control one or more of: advancement or retraction of a filament; amount of expansion of a visualization device distal portion such as the amount of radial expansion; amount of force applied to tissue walls by a visualization device; and amount of advancement or retraction of a probe such as a needle.

Referring specifically to FIG. 2a, visualization device 100a has been positioned such that target portion 129 is proximate to a target location for the advancement of a probe between artery 10 and vein 20. Target portion 129, preferably a radiopaque portion of visualization device 100, is a visualizable target used by a clinician to orient an advanceable probe, needle 212, of probe advancement device 210. As shown in FIG. 2a, needle 212 is currently contained within sheath 211 (not yet advanced).

Referring specifically to FIG. 2b, needle 212 has been slidingly advanced out of sheath 211 in the curved pathway shown, such as via a control on a handle, handle and control not shown but preferably on the proximal end of probe advancement device 210. Needle 212 may be elastically biased in the curved geometry and/or a deflecting member, not shown but within sheath 211 may deflect needle 212 in the pathway shown. Needle 212 has penetrated wall 21 of vein 20 and wall 11 of artery 10, toward target portion 129. In addition to providing a visual target for the advancement of needle 212, visualization device 100 provides measurement information such as the diameter of artery 10 via measurement of a coiled section of visualization device 100. The probe of FIGS. 2a, 2b and 2c, needle 212 exits the distal end of a shaft, sheath 211. In an alternative embodiment, a probe is configured to exit proximal to the distal end of the shaft, such as via a side hole 2-50 mm from the distal end of the shaft.

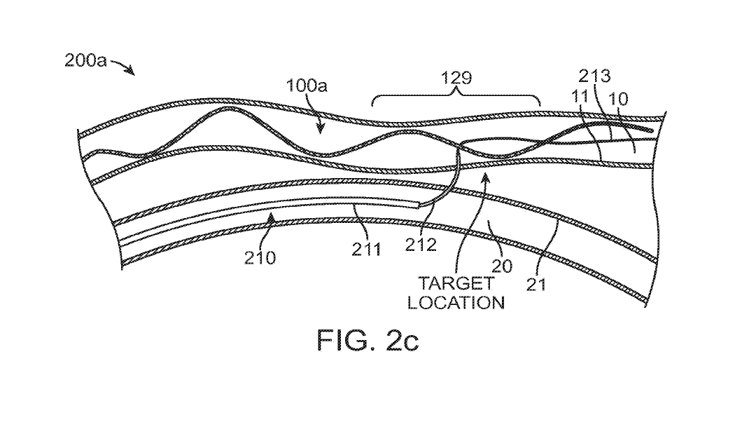

Referring specifically to FIG. 2c, guidewire 213 has been advanced through needle and into the lumen of artery 10. In subsequent steps, probe advancement device 210 can be removed leaving guidewire 213 in place, such as to act as a rail for future devices passing from vein 20 to artery 10. Such over-the-wire devices include but are not limited to: balloon catheters; anastomotic clip delivery catheters; drug deliver catheters; blood sampling catheters; radiation delivery devices; and combinations of these. In a preferred embodiment, a fistula is created and an anastomotic clip is placed, described in detail in reference to co-pending application Ser. No. 11/696,635 incorporated in its entirety herein by reference. Visualization device 100a and the other visualization devices of the present invention may be configured to allow one or more devices, such as needle 212, guidewire 213 and/or any device passing from vein 20 to artery 10, to remain in place when visualization device 100a is retracted and/or removed. During retraction, target portion 129 unwinds to prevent applying a force to any device passing through the spiral of target portion 129. In an alternative embodiment, a target or other distal portion of a visualization device may be configured to capture one or more devices passing through it, such as is described in reference to FIG. 3 herebelow.

Referring now to FIG. 3, yet another probe advancement system of the present invention is illustrated. Probe advancement system 200b includes visualization device 100b and probe advancement device 210. Visualization device 100b has been placed in artery 10, and probe advancement device 210 has been placed in vein 20, each such as via a percutaneous vessel access sheath common to interventional medical procedures. A target location, as has been described in reference to FIGS. 2a through 2c, is chosen for advancement of a probe from vein 20 to artery 10. In an alternative embodiment, a probe is advanced from an artery to a vein, from a first chamber of the heart to a second chamber of the heart, or from any location (including locations external to the patient's body) to a body space as has been defined hereabove. In a preferred embodiment, a fistula is to be created between an artery and a vein at a target location. Alternatively or additionally, the target location may be chosen to deliver drugs into artery 20, deliver radiation in artery 20 at or near the target location, and/or perform a medical procedure, including therapeutic and diagnostic procedures, at or near the target location. Visualization device 100c may include a handle on its proximal end, not shown but typically including one or more controls such as to advance and retract cage 123. Probe advancement device 210 also preferably includes a handle on its proximal end, not shown but typically including one or more controls such as to advance a probe at its distal end.

Visualization device 100b includes elongate, flexible sheath 122 which slidingly surrounds filament 127. Filament 127 includes distal portion 121 which comprises a basket design, expandable cage 123, resiliently biased in the expanded condition illustrated. Expandable cage 123 may take on various forms, including but not limited to basket, cage and stent-like geometries. Retraction of filament 127 into sheath 122, such as via a control on a handle fixedly attached to the proximal end of visualization device 100b, handle and control not shown, causes cage 123 to be captured and constrained within sheath 122. Advancement of filament 127 causes cage 123 to exit the distal end of sheath 122, radially expanding as it exits to contact luminal wall 11 of artery 10. Cage 123 is configured to be visualized under fluoroscopy or other imaging means, such that the location and geometry of the body space tissue walls, luminal walls 11 of artery 10, are clearly identified, positioned and sized. Cage 123 and other components of the present invention can have their radiopacity enhanced by placing a radiopaque coil over a portion of the device, such as over the struts of the cage. The expandable distal portions of the visualization devices of the present invention, including cage 123 of FIG. 3, may be configured to expand to a range of diameters of vessel walls, without significantly deflecting those walls or otherwise altering the normal anatomical topography. Alternatively, the distal portions may be configured to radial expand to a fixed or small range of diameters, such as to provide an enhanced radial force. Application of large radial forces may be used to assist in advancement of a probe, such as a needle, toward the distal portion (e.g. to prevent the collapse of one or more tissue walls).

Probe advancement device 210, inserted in vein 20, includes flexible sheath 211 which slidingly surrounds an advanceable probe, needle 212 shown having been advanced out of sheath 211, through venous wall 21, through arterial wall 11 and into the lumen of the artery toward cage 123. Needle 212 is advanced out of the distal end of sheath 211 in the curved geometry shown, as has been described above in reference to FIGS. 2a-2c. Once advanced into the lumen of artery 10, needle 212 can be used to perform one or more medical procedures, such as agent delivery, radiation delivery, or blood sampling, or needle 212 can be used to advance an elongate medical device, such as a guidewire, a catheter, or any therapeutic, diagnostic or other medical probe configured to pass through needle 212. In a preferred embodiment, as has been described in detail hereabove and in co-pending application Ser. No. 11/696,635 incorporated in its entirety herein by reference, a guidewire is introduced over which dilation and/or anastomosis forming devices are placed to create a long term fistula.

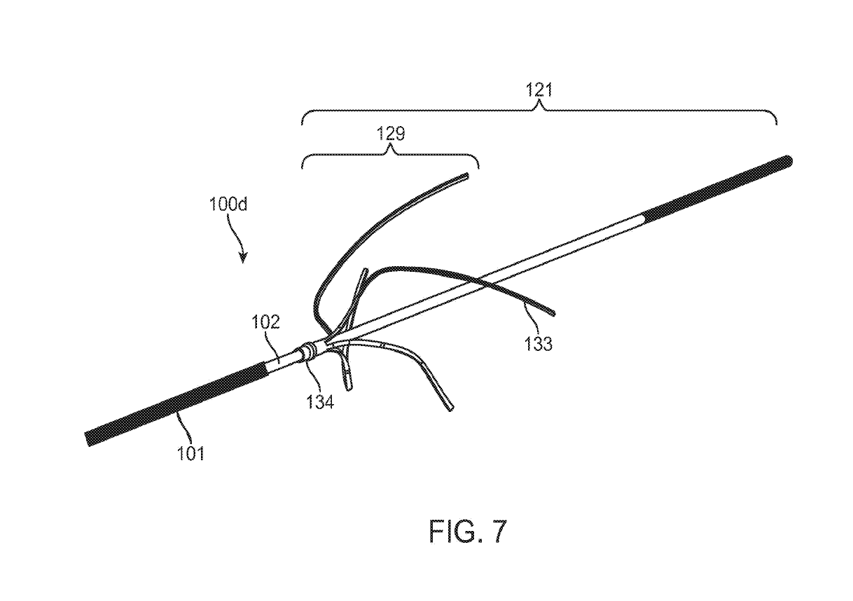

In an alternative embodiment, the distal portion of the visualization devices of the present invention, such as cage 123 of visualization device 100b, may be configured to capture an advanced probe such as an advanced needle or guidewire. Cage 123 may be partially collapsed such that a device (e.g. needle 212, a guidewire or other advanceable filament)is frictionally engaged by one or more struts of cage 123. After sufficient capture force is achieved, the advanceable probe (e.g. a guidewire) can be moved proximally or distally within artery 10 with retraction or advancement, respectively, of visualization device 100b. Capturing of an advanced probe, such as a guidewire placed from vein to artery, can be used to provide distal support when advancing a device such as a balloon catheter or anastomotic clip delivery catheter over the guidewire through tissue. In an alternative embodiment, visualization device 100b is configured to prevent capture of an advanced probe, such that visualization device 100b can be removed from the vasculature without applying force to the advanced probe. The visualization device of FIG. 7 herebelow includes an open cell design which avoids capture of a device passed there within or along the visualization device. Visualization device 100b preferably includes a handle on its proximal end, not shown but typically including one or more controls such as to advance and retract cage 123.

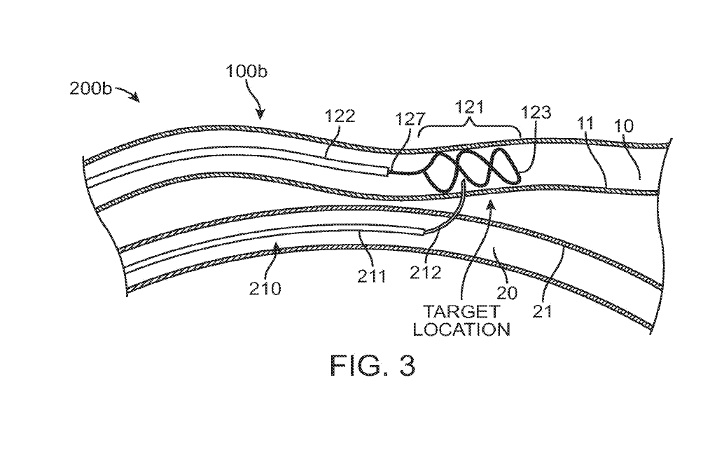

Referring now to FIGS. 4a and 4b, yet another probe advancement system of the present invention is illustrated. Probe advancement system 200c includes visualization device 100c and probe advancement device 210. Visualization device 100c has been placed in artery 10, and probe advancement device 210 has been placed in vein 20, each such as via a percutaneous vessel access sheath common to interventional medical procedures. A target location, as has been described in reference to FIGS. 2a through 2c, is chosen for advancement of a probe from vein 20 to artery 10. In an alternative embodiment, a probe is advanced from an artery to a vein, from a first chamber of the heart to a second chamber of the heart, or from any location (including locations external to the patient's body) to a body space as has been defined hereabove. In a preferred embodiment, a fistula is to be created between an artery and a vein at a target location. Alternatively or additionally, the target location may be chosen to deliver drugs into artery 20, deliver radiation in artery 20 at or near the target location, and/or perform a medical procedure, including therapeutic and diagnostic procedures, at or near the target location.

Referring specifically to FIG. 4a, visualization device 100c is shown in a near linear configuration. Visualization device 100c includes an elongate filament, hollow tube 128, which includes lumen 131. The end of hollow tube 128 is closed such that a device inserted into lumen 131 will not exit the distal end of hollow tube 128. Probe advancement device 210 is shown with needle 212 contained within a lumen of sheath 211. The distal end of sheath 211 is curved, and has not yet been oriented toward the target location in artery 10.

Referring specifically to FIG. 4b, mandrel 125, elastically biased in a helical spiral, has been advanced to the end of lumen 128. Mandrel 125 provides sufficient forces to radially expand distal portion 121 of tube 128 causing the exterior portion of distal portion 121 to contact walls 11 of artery 10 in a helical pattern. Distal portion 121, visualizable such as with a radiopaque substance visible under X-ray or fluoroscopy, can be used to size artery 10, locate a specific target site along artery 10, and provide other measurement and/or navigation functions. Mandrel 125 includes visualizable markers 126a and 126b, such as radiopaque markers that appear under X-ray at a different darkness than the other portions of distal portion 121.

The systems of the present invention preferably include one or more visualization instruments and the anatomy visualization devices, probe advancement devices and other system devices and components of the present invention are configured, at least in part, to be visualized by these visualization instruments. The systems of the present invention may be configured to work with one or more visualization systems such as X-ray systems such as fluoroscopes, ultrasound visualization systems, MRIs, and other visualization systems commonly found in healthcare centers such as hospitals. System 200c of FIG. 4b further includes visualization instrument 250 which includes fluoroscope 251 and monitor 252. Alternatively or additionally, instrument 250 may provide ultrasound images, CT-scans, MM images, PET scans, and other forms of imaging found in hospitals and healthcare centers. Fluoroscope 251 is pointed at the target site, through the Skin of the patient. Bi-plane fluoroscope may be used to create images at different orientations to the target site. Once an image of distal portion 121 is found, the distal, curved end of sheath 211 can be rotated such that as needle 212 is advanced toward the center of the lumen of artery 10. FIG. 4b illustrates probe advancement device having been oriented such that needle 212 will advance toward distal portion 121, into the center of the lumen of artery 10. Visualization device 100c preferably includes a handle on its proximal end, not shown but typically including one or more controls such as to advance and retract mandrel 125. Probe advancement device 210 preferably includes a handle on its proximal end, not shown but typically including one or more controls such as to advance needle 212.

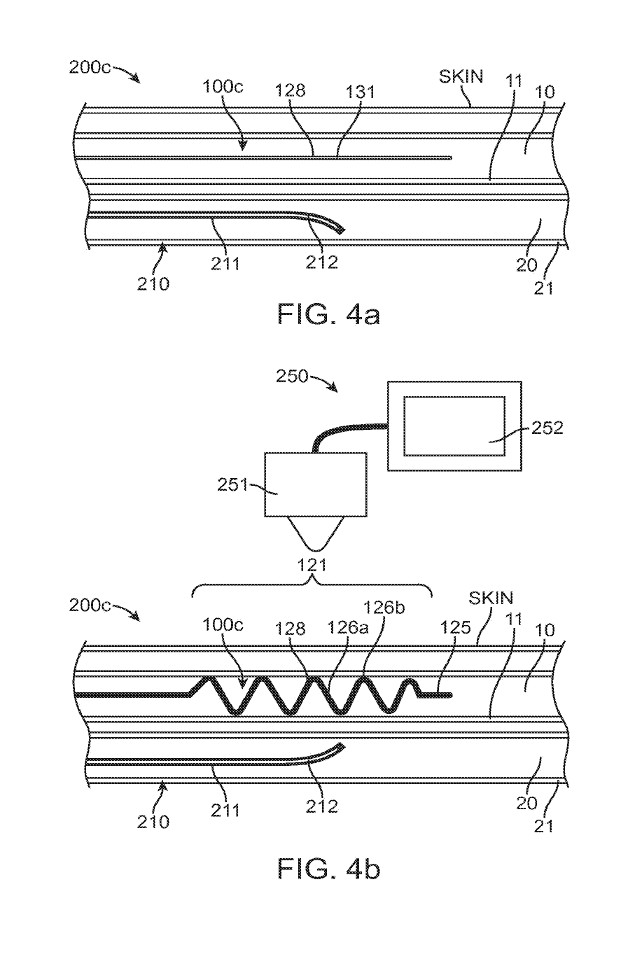

Referring now to FIG. 5, yet another probe advancement system of the present invention is illustrated. Probe advancement system 200d includes visualization device 100d and a probe advancement device, not shown but configured as has been described in reference to multiple figures hereabove. Visualization device 100d has been placed in artery 10, such as via a percutaneous vessel access sheath common to interventional medical procedures. A target location, as has been described in reference to FIGS. 2a through 2c, is chosen for advancement of a probe from vein 20 to artery 10. Visualization device 100d preferably includes a handle on its proximal end, not shown but typically including one or more controls such as to advance and retract distal portion 121.

Visualization device 100d is shown in its radially expanded state, a multiple turn helical spiral. In an alternative embodiment, partial helixes (i.e. less than 360°) can be used. Distal portion 110 may be retracted into a sheath, sheath not shown but as been described in reference to multiple figures hereabove, such that distal portion 110 is restrained in a near linear geometry. Distal portion 121, visualizable such as with a radiopaque substance visible under X-ray or fluoroscopy, can be used to size artery 10, locate a specific target site along artery 10, and provide other measurement and/or navigation functions. Additional markers may be included in distal portion 121 or at another location along visualization device 110d, such as to measure and/or located diameters of artery 10 and or longitudinal distances along artery 10.

As has been described in reference to FIGS. 2a-2c, a guidewire 213 has been placed from vein 20 to artery 10. A flexible probe device, catheter 230 has been advanced over guidewire 213, through lumen 231, such that the distal end of catheter 230 resides in the lumen of artery 10. Dilation of the vein wall 21, artery wall 11, and the tissue in-between, may have been performed (e.g. over guidewire 213 with a balloon or debulking catheter), to aid in advancement of catheter 230 into artery 10. Alternatively, catheter 230 may have a sharpened or otherwise penetrating tip, or may have an inserted penetrating element such as a sharp mandrel, all not shown. Agents delivered into a lumen of catheter 230 from its proximal end (proximal end not shown but preferably containing an attached handle with an infusion port in fluid communication with the lumen), pass around guidewire 213 and are delivered into the lumen of artery 10. Guidewire 213 may be removed during the agent delivery process, and may be replaced to remove catheter 230 leaving guidewire 230 in place. Agents, such as pharmaceutical or other liquid or solid agents, may be delivered for site specific delivery at the location of distal portion 121. In an alternative embodiment, other matter such as magnetic particles or implantable medical devices may be delivered through the lumen of catheter 230. After the proper amounts of agents have been delivered, catheter 230 is removed. Guidewire 213 may also be removed at that time, or left in place for insertion of another over-the-wire device.

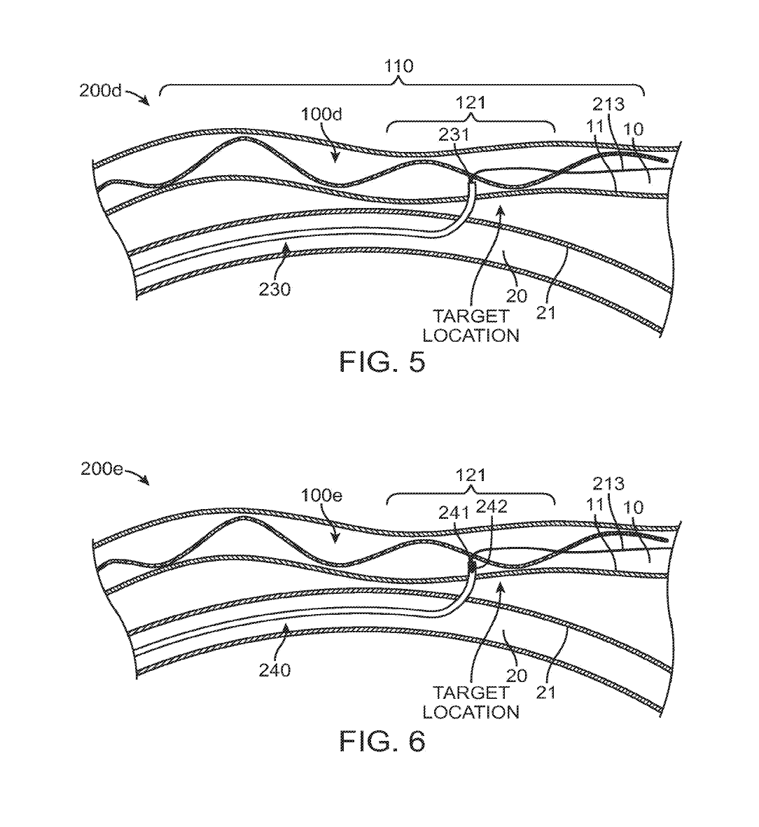

Referring now to FIG. 6, yet another probe advancement system of the present invention is illustrated. Probe advancement system 200e includes visualization device 100e and a probe advancement device, not shown but configured as has been described in reference to multiple figures hereabove. Visualization device 100e has been placed in artery 10, such as via a percutaneous vessel access sheath common to interventional medical procedures. A target location, as has been described in reference to FIGS. 2a through 2c, is chosen for advancement of a radioactive probe from vein 20 to artery 10. Visualization device 100e preferably includes a handle on its proximal end, not shown but typically including one or more controls such as to advance and retract distal portion 121.

Visualization device 100e is shown in its radially expanded state, a multiple turn helical spiral. In alternative embodiment, partial helixes (i.e. less than 360°) can be used. Distal portion 110 may be retracted into a sheath, sheath not shown but as been described in reference to multiple figures hereabove, such that distal portion 110 is restrained in a near linear geometry. Distal portion 121, visualizable such as with a radiopaque substance visible under X-ray or fluoroscopy, can be used to size artery 10, locate a specific target site along artery 10, and provide other measurement and/or navigation functions. Additional markers may be included in distal portion 121 or at another location along visualization device 110e, such as to measure and/or located diameters of artery 10 and or longitudinal distances along artery 10.

As has been described in reference to FIGS. 2a-2c, guidewire 213 has been placed from vein 20 to artery 10. A flexible probe device, radiation delivery device 240 has been advanced over guidewire 213, through lumen 241, such that the distal end of catheter 230 resides in the lumen of artery 10. Dilation of the vein wall 21, artery wall 11, and the tissue in-between, may have been performed (e.g. over guidewire 213 with a balloon or debulking catheter), to aid in advancement of radiation delivery device 240 into artery 10. Alternatively, device 240 may have a sharpened or otherwise penetrating tip, or may have an inserted penetrating element such as a sharp mandrel, all not shown. Radiation delivery device 240 includes radioactive element 242 near its distal end, positioned within the lumen of artery 10. Radiation delivery device 240 remains in place for a time sufficient to deliver the radioactive energy, such as to treat and/or prevent neointimal proliferation or other vessel narrowing at the target location. After the proper amount of radiation has been delivered, radiation delivery device 240 is removed. Guidewire 213 may also be removed at that time, or left in place for insertion of another over-the-wire device.

Referring now to FIG. 7, another embodiment of an anatomy visualization device of the present invention is illustrated. Anatomy visualization device 100f includes distal portion 121. Distal portion 121 includes target portion 129 comprising multiple filaments, tines 133, configured in an open cell design, such as to avoid capturing one or more filaments or other devices advanced toward target portion 129. The proximal ends of tines 133 are fixedly attached to shaft 102 and distal portion 121 by band 134. Alternatively, tines 133 may be cut from a single tube integral to distal portion 121. Target portion 129 is shown having exited the distal end of sheath 101 (e.g. via retraction of sheath 101) causing the distal ends of tines 133 to extend radially outward, such as to contact the walls of a body space such as an artery or a vein. Tines 133 may be recaptured such as via retraction of shaft 102 and/or advancement of sheath 101. Tines 133 are made of a visualizable material or include one or more visualizable markers, not shown. In a preferred embodiment, tines 133 include a radiopaque material or include one or more radiopaque markers, such that tines 133 can be visualized under fluoroscopy or other x-ray equipment to assist a clinician in locating a body space or a portion of a body space.

It should be understood that numerous other configurations of the devices, systems and methods described herein can be employed without departing from the spirit and scope of this application. Numerous figures have illustrated typical dimensions, but it should be understood that other dimensions can be employed which result in similar functionality and performance. The devices and systems of the present invention may be used to perform various procedures including medical procedures such as the creation of an arteriovenous fistula.

The anatomy visualization devices of the present invention include a target portion which is used to direct the advancement of an advanceable probe such as an advanceable needle and/or a guidewire. The target portion may be located at any location along the device, typically near the distal end. Target portion may be self-expanding, or may be expanded by mechanical or other means, such as via introduction of a shaped mandrel. The anatomy visualization devices of the present invention may be configured to be removed while the advanceable probe is located within or alongside the target portion, such as to leave the probe in place for a subsequent action or procedural step. Alternatively, the anatomy visualization devices of the present invention may be configured to capture or otherwise apply a retaining force to a probe advanced near or through the anatomy visualization device, such as to maintain position of the advanceable probe during a subsequent action or procedural step.

The devices described above may include one or more markers, such as radiopaque, ultrasonic, magnetic or other visualizable markers, to assist in visualizing the device during use. The entire device may be radiopaque or one or more portions, such as the target portion, are radiopaque. The device may include one portion with a first radiopacity, and a second portion with a radiopacity different than the first radiopacity, such as to distinguish one portion from another during a medical procedure. Radiopaque coatings or coils may include materials such as tungsten, platinum, gold, and/or a doped polymer such as a polymer including barium sulfate. The devices described above may be provided with coatings or additional structures which serve as matrices for various therapeutic compounds. Drug eluting coatings, additional drug eluting members, drug eluting membranes surrounding tubular sections or drug eluting masses that fill integrated chambers or wells may be added to the devices.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims.

What is claimed is:

1. A method of creating a fistula between a first vessel and a second vessel, the method comprising:
placing a probe advancement device into the first vessel;
determining a diameter of the second vessel at a target site;
selecting an anatomy visualization device having a target portion configured to self-expand to a diameter larger than the determined diameter of the second vessel and to a configuration with spiral geometry, said anatomy visualization device comprising a single elongate filament comprising the target portion which is constructed of or coated with a material that can be visualized by a visualization instrument;
advancing the anatomy visualization device through the second vessel so that the target portion is positioned at the target site of the second vessel;
allowing the target portion to self-expand toward one or more tissue walls of the second vessel to directly contact said one or more tissue walls at the target site;
visualizing self-expanded target portion contacting said one or more tissue walls with the visualization instrument; and
advancing the probe advancement device toward said target portion of the anatomy visualization device and into the target site of the second vessel as guided by the visualizing of the self-expanded target portion, thereby creating the fistula.

2. The method of claim 1, wherein the probe advancement device comprises an elongate tube and an advanceable probe positioned therewithin, and wherein advancing the probe advancement device comprises advancing the advanceable probe through the elongate tube.

3. The method of claim 1, wherein the self-expanding target portion comprises one or more markers visible to the visualization instrument.

4. The method of claim 1, wherein the self-expanding target portion has greater visibility to the visualization instrument than a portion of the single elongate filament proximal thereto.

5. The method of claim 1, further comprising advancing the probe advancement device to a position adjacent the target site of the second vessel.

6. The method of claim 1, further comprising retracting the anatomy visualization device in the second vessel after the fistula has been created.

7. The method of claim 1, further comprising advancing a further device through the probe advancement device and into the second vessel after the fistula has been created.

8. The method of claim 7, wherein the further device comprises an anastomotic clip.

9. The method of claim 1, wherein the visualization instrument is selected from the group consisting of: a fluoroscope or other x-ray visualization apparatus, an ultrasound visualization apparatus, a computed tomography (CT) scanner, a magnetic resonance imaging (MM) apparatus, a positron emission tomography (PET) scanner, an electromagnetic (EM) field detection apparatus, and combinations thereof

10. The method of claim 1, wherein the diameter of the second vessel is determined with the visualization apparatus.

11. The method of claim 1, wherein the single elongate element is flexible.

12. The method of claim 1, wherein the single elongate element is constructed of materials selected from the group consisting of: Nitinol, stainless steel, tungsten, cobalt chromium (CoCr), a polymer or polymer blend, and combinations thereof.

13. The method of claim 1, wherein the single elongate filament includes a coating selected from the group consisting of: a hydrophobic coating, a hydrophilic coating, a polytetrafluoroethylene (PTFE) coating, a polytetrafluoroethylene (PTFE) covering, and combinations thereof

14. The method of claim 1, wherein the target portion comprises a marker selected from the group consisting of: a radiopaque marker, an electromagnetic marker, a magnetic marker, an ultrasonically reflective marker, and combinations thereof.

15. The method of claim 1, wherein allowing the target portion to self-expand comprises allowing the self-expanding target portion to radially expand outward from a central axis.

16. The method of claim 1, wherein the single elongate element further includes a straight portion distal of the target portion.

17. The method of claim 1, wherein allowing the target portion to self-expand comprises removing a constraint from the target portion.

18. The method of claim 17, wherein removing the constraint comprises retracting a sheath of the anatomy visualization device relative to the target portion.

19. The method of claim 1, wherein allowing the target portion to self-expand toward one or more tissue walls to directly contact said one or more tissue walls comprises allowing the self-expanded target portion to exert a radially outward force to the one or more tissue walls.

20. The method of claim 1, wherein the anatomy visualization device is selected so that the target portion is oversized compared to the target site of the second vessel by 1-2 mm.

21. The method of claim 1, wherein the diameter of the single elongate filament at the target portion is smaller than at straight portions of the single elongate filament so as to reduce radial force exerted by the target portion on the target site of the second vessel when self-expanded therein.

22. The method of claim 1, wherein the target portion is configured to be elastically biased to self-expand.

23. The method of claim 1, wherein the target portion is configured to be temperature activated to self-expand.

24. The method of claim 23, wherein the temperature activation occurs at body temperature.

25. The method of claim 1, wherein the target portion is configured to be mechanically activated to self-expand.

26. The method of claim 1, wherein the spiral configuration of the target portion includes more than 360° of spiral.

27. The method of claim 1, wherein the target portion when self-expanded has a diameter of at least 2 mm.

28. The method of claim 1, wherein the probe advancement device is placed into the first vessel percutaneously.

29. The method of claim 1, wherein the second vessel comprises a body space selected from the group consisting of: a blood vessel lumen, a chamber of the heart, a stomach, a urethra, or a biliary duct.
